(19) United States
(12) Patent Application Publication
(10) Pub. No.: US 2005/0267420 A1
(43) Pub. Date: Dec. 1, 2005

(54) Protective device for a needle assembly and assembly comprising same

(76) Inventor: Christopher S. Young, South Kent, CT (US)

Correspondence Address: Arthur G. Schaier;Carmody & Torrance LLP, 50 Leavenworth Street, P.O. Box 1110, Waterbury, CT, 06721-1110, US

(21) Appl. No.: 10/853,341

(22) Filed: May 25, 2004

(51) Int. Cl. A61M 5/00

(52) U.S. Cl. 604/263

(57) ABSTRACT

A protective device for a needle assembly. The protective device comprises an elongated member having a distal end and a second end; a cap, coupled to the distal end of the elongated member, including a recess therein for receiving the distal end of the cannula; a hub coupler, coupled to the second end of the elongated member, for coupling to the hub of the needle assembly; wherein the protective device is dimensioned so that when the distal end of the cannula is positioned in the recess of the cap, the hub coupler is couplable to the hub of the needle assembly. A method of using the protective needle device with a needle assembly is also provided and comprises the steps of positioning the distal end of the cannula in the recess of the cap; rotating the protective device and the needle assembly towards each other so as to bring the hub coupler into close proximity with the hub; and coupling the hub coupler to the hub.

### BACKGROUND OF THE INVENTION

The present invention relates generally to needle assemblies, and in particular, to a protective device for a needle assembly that reduces the likelihood of inadvertent needle sticks while also facilitates the ability to reposition the protective device over the needle after a procedure, such as if someone desires to use one hand in doing so.

Needle assemblies for use in medical procedures or operations in which blood or other fluids must be drawn from a patient or injected into a patient, or in which bone, tissue or tumors are removed, or in which catheters (or the like) are positioned in a body, are well known. Many of these devices are designed with the recognition that protection of the doctor (or other authorized medical personnel) from being inadvertently “stuck” by the needle (or cannula) is important.

Unfortunately, other than the technology developed by the Assignee of the present invention (i.e. that which is described in U.S. Pat. No. 6,475,190), the present state of the art is generally deficient in the foregoing regard. For example, because most known “needle shields” are removable, the needle is dangerously exposed after it is used. This leaves open the possibility that the needle can still cause an undesirable “poking” or “sticking.” Therefore, a needle guard or shield that is easily replaceable over the needle is desired.

Accordingly, it is desirable to provide an improved protective device and assembly that overcomes the perceived deficiencies in the prior art noted above and further achieves the aforementioned and below mentioned objectives; namely, an improved protective device for a needle assembly that can be used by medical personnel or other appropriate/necessary users, and that is both effective and protective of everyone that would handle the needle assembly.

### SUMMARY AND OBJECTIVES OF THE INVENTION

Generally speaking, in accordance with the present invention, an improved protective device for a needle assembly is provided. In a preferred construction, the protective device comprises an elongated member having a distal end and a second end; a cap, coupled to the distal end of the elongated member, including a recess therein for receiving the distal end of the cannula; a hub coupler, coupled to the second end of the elongated member, for coupling to the hub of the needle assembly; wherein the protective device is dimensioned so that when the distal end of the cannula is positioned in the recess of the cap, the hub coupler is couplable to the hub of the needle assembly.

Specific features of the present invention provide that when the distal end of the cannula is positioned in the recess of the cap, the protective device is pivotable about the distal end of the cannula. In other words, the protective device is dimensioned to rotate towards the needle assembly so as to bring the hub coupler into close proximity with the hub and permit the coupling the hub coupler to the hub. Here, the distal end of the cannula is preferably in frictional engagement with an inner surface of the recess when the hub coupler is couplable to the hub.

In a preferred embodiment, the elongated member is positioned adjacent the cannula so as to expose at least a portion of the cannula when the distal end of the cannula is positioned in the recess of the cap and the hub coupler is coupled to the hub. In a specific embodiment, the hub coupler is snap-fittedly couplable to the hub such as by the use of resilient fingers on the hub coupler. Additionally, it is preferred that the elongated member is tubular and dimensioned to receive the cannula therein, and further wherein the hub coupler is couplable to the hub when the cannula is positioned within the elongated member, whereby when the cannula is positioned within the elongated member and the hub coupler is coupled to the hub, the entire length of the cannula is protected within the elongated member.

In this manner, an assembly that includes both the needle assembly and the protective device is provided.

Lastly, a method of using the protective needle device with the needle assembly is also provided. Here, the method comprises the steps of positioning the distal end of the cannula in the recess of the cap; and rotating the protective device and the needle assembly towards each other so as to bring the hub coupler into close proximity with the hub; and coupling the hub coupler to the hub.

If the hub coupler comprises a first finger and a second finger spaced apart from the first finger, the method may comprise the steps of snap-fitting the first and second fingers around a portion of the hub whereby the protective device is releasably secured to the needle assembly. Similarly, if the elongated member is tubular, it may be dimensioned to receive the cannula therein such that the method includes the prior steps of positioning the cannula within the elongated member and coupling the hub coupler to the hub so that the entire length of the cannula is protected within the elongated member; and thereafter, removing the cannula from within the elongated member; and performing a procedure using the needle assembly.

Accordingly, it is an objective of the present invention to provide an improved protective device for a needle assembly.

Another objective of the present invention is to provide an improved assembly comprising a needle assembly and protective device that is safer to use by reason of its inclusion of an improved construction for covering the distal end of the cannula after use.

Yet another objective of the present invention is to provide an improved protective device for use with a needle assembly that can be repositioned over the cannula after use.

Still another objective of the present invention is to provide an improved protective device that achieves all of the aforementioned objectives that is both easy and inexpensive to manufacture.

Still another objective of the present invention is to provide a method of using a protective device constructed in accordance with the present invention with commercially available needle assembly constructions.

Still other objects and advantages of the invention will in part be obvious and will in part be apparent from the specification.

The invention accordingly comprises the features of construction, combination of elements, arrangement of parts and sequence of steps which will be exemplified in the construction, illustration and description hereinafter set forth, and the scope of the invention will be indicated in the claims.

### BRIEF DESCRIPTION OF THE DRAWINGS

For a fuller understanding of the invention, reference is had to the following description taken in connection with the accompanying figures, in which:

FIG. 1 is a perspective view of a protective device for a needle assembly, constructed in accordance with the present invention;

FIG. 2 illustrates an interim step in the methodology of the present invention, namely, the step of inserting the distal end of the cannula into the cap of the protective device, such step preferably occurring after the needle assembly has been used and is ready to be discarded;

FIG. 3 a perspective view of the protective device of FIG. 1, after the step of FIG. 2 is performed, being in its secured and releasably locked position with the needle assembly;

FIG. 4 is an enlarged perspective view of a portion of the protective device and needle assembly illustrated in FIG. 3;

FIG. 5 is a perspective view of an assembly comprising the needle assembly and a protective device constructed in accordance with the present invention, all in the assembled configuration prior to use; and

FIG. 6 illustrates an interim step in the methodology of the present invention, namely the step of removing the needle assembly from the protective device prior to the using of the needle assembly in a medical procedure.

Also, while not all elements are labeled in each figure, all elements with the same reference number indicated like parts.

### DETAILED DESCRIPTION OF THE PREFERRED EMBODIMENTS

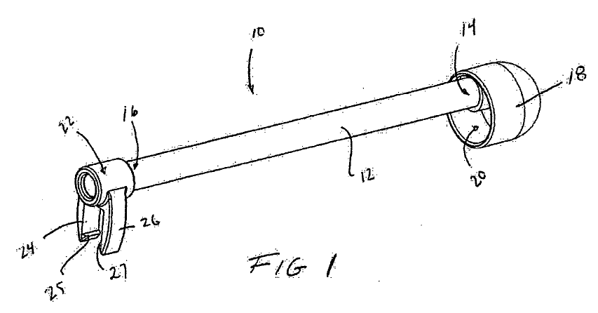

Reference shall now be made to the Figures, and first to FIG. 1, wherein a protective device, generally indicated at 10, constructed in accordance with the present invention, is disclosed. Protective device 10 is preferably used with a needle assembly that will be described in greater detail below.

Specifically, protective device 10 comprises an elongated member 12 having a distal end, generally indicated at 14 and a second end, generally indicated at 16. A cap 18 is coupled to distal end 14. As can be seen in FIG. 1, cap 18 includes a recess 20 therein for receiving the distal end of the cannula of the needle assembly that will be described below. A hub coupler, generally indicated at 22, is coupled to second end 16 of elongated member 12. As will be described below, hub coupler 22 is for coupling protective device 10 to the hub of the needle assembly. Protective device 10 is dimensioned so that when the distal end of the cannula is positioned in recess 20 of cap 18, hub coupler 22 is couplable to the hub of the needle assembly.

In order to further and better describe the preferred construction of the protective device, a brief description of the needle assembly shall now be described. Generally speaking, a preferred needle assembly for use with the present invention is described in U.S. patent application Publication No. U.S. 2002/0055715, and therefore, the subject matter of this application is incorporated by reference as if fully set forth herein. However, for the ease of the reader, the following is set forth.

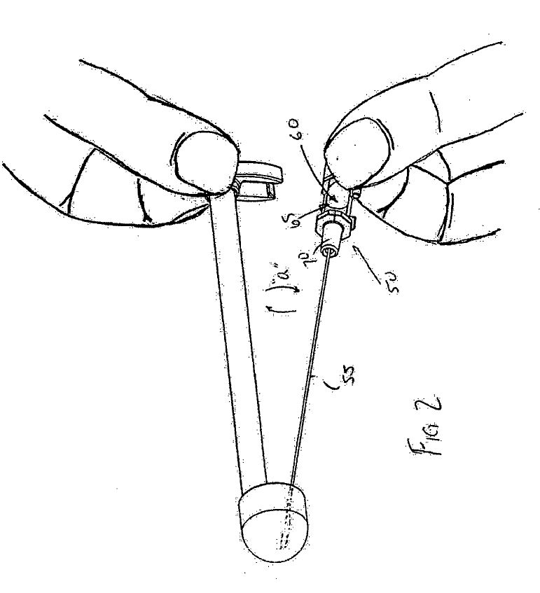

The preferred needle assembly, illustrated (at least partly) in each of the remaining FIGS. 2-6, is generally indicated at 50, and generally speaking, is comprised of a cannula 55 and a hub, generally indicated at 60.

In the preferred embodiment so that even the needle assembly itself can appreciate all the advantages and objectives set for in the aforementioned Publication No. U.S. 2002/0055715, hub 60 includes a hub body portion, generally indicated at 65, with an internal hollow region (not shown) in which blood or other fluids may be collected. However, the present invention is equally applicable to a needle assembly in which fluid is discharged, such as but not limited to a syringe. Likewise, the present invention is equally applicable to a needle assembly in which bone, tissue and/or tumors are collected as would be understood in the art. The hollow region is also preferably magnified in a manner as described in the aforementioned application. Hub 60 further includes a neck portion 70 preferably integrally formed with body portion 65, with neck portion 70 having a bore (not shown) therethrough that opens at a first end thereof into the hollow region in body portion 65.

On the other hand, but as would also be clearly understood, cannula 55 has two ends, a distal end 56 (which may be tapered) and a second end which is somewhat hidden from view since it has been, consistently depicted in the Figures as being positioned in an end of the bore of neck portion 70, all in a manner as would be well understood in the art and which forms no part of the invention. With the second end of cannula 55 positioned in neck portion 70, blood or other fluids designed to enters tip 56 of cannula 55 during a medical procedure can enter the hollow region (e.g. flash chamber) of body portion 65 in a known manner. Cannula 55 is preferably made of stainless steel and has a pointed, beveled or conically shaped end with a side port entry. Cannula 55 may be affixed to the hub via either medical grade epoxy or insert molding to meet the pull test requirements of ISO 594.

The two side surfaces 61, 62 of hub 60 may be concave in shape so as to provide increased finger grip for easy hub positioning during needle placement. As will be disclosed below, such concave side surfaces may also provide the region on the hub to which hub coupler 22 will be coupled.

With reference now to FIG. 2, and returning to the particulars of the protective device 10, when the distal end 56 of cannula 55 is positioned in recess 20 of cap 18, protective device 10 is pivotable about distal end 56 of cannula 55. In other words, the protective device 10 and the needle assembly are rotatable towards each other as shown by the arrows “a”. More specifically, the distal end of cannula 55 is preferably in frictional engagement with an inner surface of recess 20 such as that noted by the dotted lines in FIG. 2. In fact, cap 18 may include an interior dimpled recess (not shown) to further facilitate the positioning of distal end 56 of cannula 55 in recess 20 of cap 18. In a similar way, cap 18 may be bullet-shaped to still further guide the distal end of cannula 55 to the apex of recess 20.

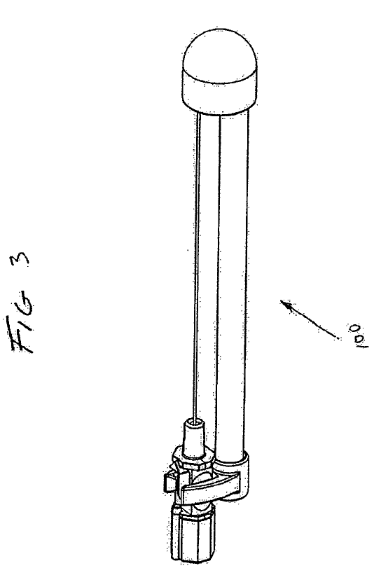

FIG. 3 illustrates protective device 10 secured to needle assembly 50 after use thereof. Protective device 10 is preferably dimensioned so that when distal end 56 of cannula 55 is positioned in recess 20 of cap 18, hub coupler 22 is couplable (and in FIG. 3 is actually coupled) to hub 60 of needle assembly 50. More specifically, when distal end 56 of cannula 55 is positioned in recess 20 of cap 18 and hub coupler 22 is coupled to hub 60, elongated member 12 is positioned adjacent cannula 55 so as to expose at least a portion of cannula 55. In this configuration, FIG. 3 illustrates a fully assembled assembly 100.

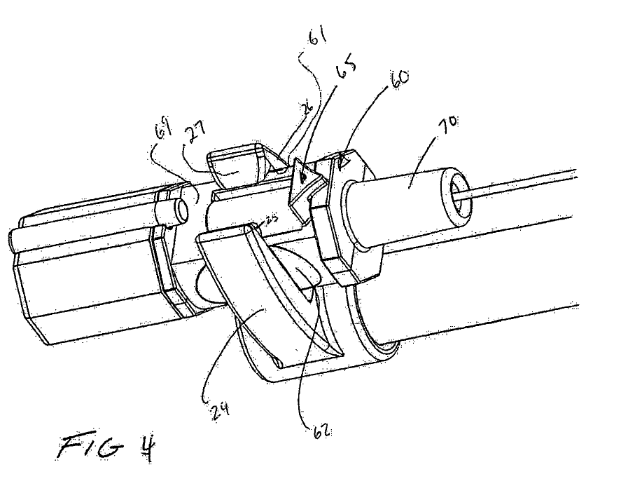

With reference to FIG. 4, hub coupler 22 is preferably snap-fittedly couplable to hub 60. To achieve this, hub coupler 22 preferably comprises a first finger 24 (with a depending flange 25) and a second finger 26 (with its own respective depending flange 27) spaced apart from first finger 24. Here, and as illustrated, first and second fingers 24, 26 snap-fit around a portion of the hub, preferably at about the respective concave side surfaces 62, 61. Depending flanges can releasably grasp onto the top edge surface 69 of hub body 65.

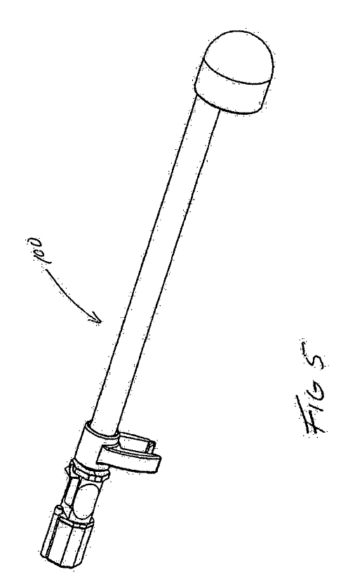

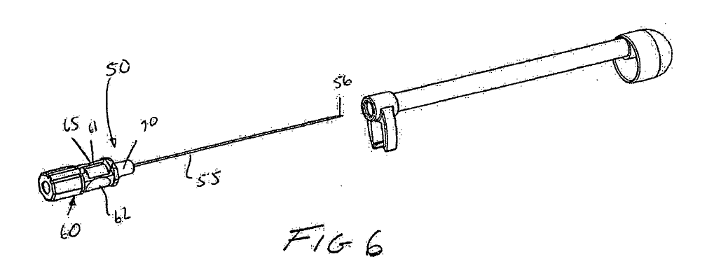

Reference is now made to FIGS. 5 and 6 wherein other features of construction of the preferred protective device is illustrated. For example, preferably elongated member 12 is tubular, and dimensioned to receive cannula 55 therein (FIG. 5). Furthermore, hub coupler 22 is constructed to be couplable to the neck portion 70 of hub 60 (such as by friction fit) when cannula 55 is positioned within elongated member 12. Here, when cannula 55 is positioned within elongated member 12 and hub coupler 22 is coupled to hub 60, the entire length of the cannula is protected within the elongated member. This arrangement provides for the preferred shipping and/or storing configuration (e.g. prior to the needle assembly's use in a medical procedure). Also, in this way, there is a distinct visual difference between an unused and a used needle assembly 50. Thereafter, one merely has to remove cannula 55 from within elongated member 12 (FIG. 6) and perform a procedure (not shown) using the needle assembly.

As can thus be seen, an assembly 100 that comprises needle assembly 50 and protective device 10 is thus provided.

After cannula 55 is used for its intended purpose, the person (e.g. medical doctor or the like) need only guide the distal end of cannula 55 into the recess of cap 18 and thereafter, releasably lock hub coupler 22 to hub 60. More specifically, the method of using protective needle device 10 with a needle assembly, such as that exemplary illustrated in the figures, comprises the steps of positioning the distal end 56 of cannula 55 in recess 20 of cap 18 and causing the end 56 of the cannula to find and rest at the apex of the recess; rotating the protective device and the needle assembly towards each other (in the direction of arrow “a” of FIG. 2) so as to bring hub coupler 22 into close proximity with hub 60; and coupling hub coupler 22 to hub 60. Where hub coupler 22 comprises, fingers 24, 26, the method comprises the steps of snap-fitting fingers 24, 26 around the body of hub 60. In particular, but only exemplarily and not limiting, the construction of concave side surfaces 61, 62 facilitate the use of fingers 24 and 26. Moreover, depending flanges 25 and 27 assist in grasping hub 60 as illustrated in FIG. 4. In this manner, protective device 10 is releasably secured to needle assembly 50 and can be safely discarded. It should also be understood that hub coupler 22 could be replaced by other types of securing means, such as Velcro or other types of locking configurations, all of which would be well-known to the ordinarily skilled artisan.

It will thus be seen that the objects set forth above, among those made apparent from the preceding description, are efficiently attained and, since certain changes may be made in the above constructions and methodologies without departing from the spirit and scope of the invention, it is intended that all matter contained in the above description or shown in the accompanying drawings shall be interpreted as illustrative and not in a limiting sense.

It is also to be understood that the following claims are intended to cover all of the generic and specific features of the invention described herein and all statements of the scope of the invention that as a matter of language might fall therebetween.

For example, hub body 65 need not comprise concavely shaped outerside surfaces 61, 62 to facilitate the gripping thereof by a use's fingers. Such a construction would only then require somewhat more resilient fingers 24, 26 to get around the bulging outer surface of hub body 65. Additionally, hub 60 is preferably made from polycarbonate as is well known in the art while cannula 55 is also made from known conventional materials. Additionally, protective device 10 is preferably made from low-density polypropylene so as to provide the needed flexibility as set forth hereinabove. Moreover, while the present embodiment implies separated constructed elements (i.e. hub coupler 22, elongated member 12 and cap 18), clearly, an integrally molded one-piece construction is contemplated herein. Also, the construction of protective device 10 is intended to work well with any length and/or type of needles—spinal, biopsy, epidural, etc., and therefore, the length and/or type of needle should not be a limiting factor in the interpretation and or appreciation of the scope of the claims.

While the invention has been particularly shown and described with respect to preferred embodiments thereof, it will be understood by those skilled in the art that changes in form and details may be made therein without departing from the scope and spirit of the invention.

1. A protective device for a needle assembly, wherein the needle assembly comprises a cannula and a hub, and further wherein the cannula comprises a distal end and a second end that is couplable to a first end of the hub so that fluid is flowable through the distal end of the cannula, the second end of the cannula and into and/or out of the hub, wherein the protective device comprises:
an elongated member having a distal end and a second end;
a cap, coupled to the distal end of the elongated member, including a recess therein for receiving the distal end of the cannula; and
a hub coupler, coupled to the second end of the elongated member, for coupling to the hub of the needle assembly;
wherein the protective device is dimensioned so that when the distal end of the cannula is positioned in the recess of the cap, the hub coupler is couplable to the hub of the needle assembly.

2. The protective device as claimed in claim 1, wherein when the distal end of the cannula is positioned in the recess of the cap, the protective device is pivotable about the distal end of the cannula.

3. The protective device as claimed in claim 2, wherein the distal end of the cannula is in frictional engagement with an inner surface of the recess when the hub coupler is couplable to the hub.

4. The protective device as claimed in claim 1, wherein the elongated member is positioned adjacent the cannula so as to expose at least a portion of the cannula when the distal end of the cannula is positioned in the recess of the cap and the hub coupler is coupled to the hub.

5. The protective device as claimed in claim 1, wherein the cap includes an interior dimpled recess to further facilitate the positioning of the distal end of the cannula in the recess of the cap.

6. The protective device as claimed in claim 1, wherein the hub coupler is snap-fittedly couplable to the hub.

7. The protective device as claimed in claim 6, wherein the hub coupler comprises a first finger and a second finger spaced apart from the first finger, wherein the first and second fingers snap-fit around a portion of the hub.

8. The protective device as claimed in claim 1, wherein the elongated member is tubular, and wherein the elongated member is dimensioned to receive the cannula therein, and further wherein the hub coupler is couplable to the hub when the cannula is positioned within the elongated member;
whereby when the cannula is positioned within the elongated member and the hub coupler is coupled to the hub, the entire length of the cannula is protected within the elongated member.

9. An assembly, the assembly comprising:
a needle assembly comprising:
a hub comprising a body portion with an internal hollow region in which fluid is one of collected or discharged and a neck portion having a bore therethrough that opens at a first end thereof into the hollow region in the body portion; and
a cannula couplable to the hub, the cannula comprising a distal end and a second end which is insertable into an other end of the bore of the neck portion; and
a protective device, the protective device comprising:
an elongated member having a distal end and a second end;
a cap, coupled to the distal end of the elongated member, including a recess therein for receiving the distal end of the cannula; and
a hub coupler, coupled to the second end of the elongated member, for coupling to the hub of the needle assembly;
wherein the protective device is dimensioned so that when the distal end of the cannula is positioned in the recess of the cap, the hub coupler is couplable to the hub of the needle assembly.

10. The assembly as claimed in claim 9, including the dimensioning the protective device to permit the rotating of the protective device and the needle assembly towards each other so as to bring the hub coupler into close proximity with the hub and permit the coupling the hub coupler to the hub.

11. The assembly as claimed in claim 9, wherein when the distal end of the cannula is positioned in the recess of the cap, the protective device is pivotable about the distal end of the cannula.

12. The assembly as claimed in claim 9, wherein when the distal end of the cannula is positioned in the recess of the cap and the hub coupler is coupled to the hub, the elongated member is positioned adjacent the cannula so as to expose at least a portion of the cannula.

13. The assembly as claimed in claim 9, wherein the hub coupler comprises a first finger and a second finger spaced apart from the first finger, wherein the first and second fingers are snap-fittable to the body of the hub.

14. The assembly as claimed in claim 12, wherein the elongated member is tubular, and wherein the elongated member is dimensioned to receive the cannula therein, and further wherein the hub coupler is couplable to the neck of the hub when the cannula is positioned within the elongated member;
whereby when the cannula is positioned within the elongated member and the hub coupler is coupled to the hub, the entire length of the cannula is protected within the elongated member.

15. A method of using a protective needle device with a needle assembly, wherein the needle assembly comprises a hub and a cannula having a distal end and a second end that is couplable to a first end of the hub, and the protective needle device comprises an elongated member having a distal end and a second end; a cap, coupled to the distal end of the elongated member, including a recess therein for receiving the distal end of the cannula; and a hub coupler, coupled to the second end of the elongated member, for coupling to the hub of the needle assembly; wherein the method comprises the steps of:
positioning the distal end of the cannula in the recess of the cap;
rotating the protective device and the needle assembly towards each other so as to bring the hub coupler into close proximity with the hub; and
coupling the hub coupler to the hub.

16. The method as claimed in claim 15, wherein the hub coupler comprises a first finger and a second finger spaced apart from the first finger, and wherein the method comprises the step of:
snap-fitting the first and second fingers around a portion of the hub;
whereby the protective device is releasably secured to the needle assembly.

17. The method as claimed in claim 15, wherein the elongated member is tubular, and wherein the elongated member is dimensioned to receive the cannula therein, and further wherein the hub coupler is couplable to the hub when the cannula is positioned within the elongated member; wherein and prior to performing the steps recited in claim 15, performing the steps of:
positioning the cannula within the elongated member and coupling the hub coupler to the hub so that the entire length of the cannula is protected within the elongated member; and thereafter,
removing the cannula from within the elongated member; and
performing a procedure using the needle assembly.
